(19) United States
(12) Patent Application Publication
(10) Pub. No.: US 2024/0291404 A1
(43) Pub. Date: Aug. 29, 2024

(54) VIBRATION SELF-GENERATING BATTERY OF WATER QUALITY LOW-POWER SENSOR APPLICABLE TO MULTIPLE SCENARIOS

(71) Applicant: China Agricultural University, Beijing (CN)

(72) Inventors: Yang Wang, Beijing (CN); Feiyu Zhu, Beijing (CN); Shanhong Zhang, Beijing (CN); Quanwu Ge, Beijing (CN); Guanghui Yu, Beijing (CN); Fu Chai, Beijing (CN); Daoliang Li, Beijing (CN)

(73) Assignee: China Agricultural University, Beijing (CN)

(21) Appl. No.: 18/413,460

(22) Filed: Jan. 16, 2024

(30) Foreign Application Priority Data
Feb. 28, 2023 (CN) 202310175275.8

(51) Int. Cl. H02N 1/04; G01N 33/18; H02J 7/00; H02K 35/02

(52) U.S. Cl. CPC H02N 1/04; G01N 33/1886; H02J 7/0042; H02K 35/02

(57) ABSTRACT

A vibration self-generating battery of a water quality low-power sensor applicable to multiple scenarios, belonging to the technical field of self-generating energy supply. The battery includes a positive electrode magnetic cap, a battery outer cylinder, a negative electrode magnetic cap, a circuit module, a supporting component, an electromagnetic induction generating unit, and a friction nano generating unit. The positive electrode magnetic cap is arranged on the upper side of the battery outer cylinder, and the negative electrode magnetic cap is arranged at the lower side of the battery outer cylinder. The friction nano generating unit is arranged close to an inner wall of the battery outer cylinder. The electromagnetic induction generating unit is arranged at the center of the battery, and the supporting component and the circuit module are arranged below the electromagnetic induction generating unit in turn.

### CROSS-REFERENCE TO RELATED APPLICATION

This patent application claims the benefit and priority of Chinese Patent Application No. 2023101752758, filed with the China National Intellectual Property Administration on Feb. 28, 2023, the disclosure of which is incorporated by reference herein in its entirety as part of the present application.

### TECHNICAL FIELD

The present disclosure relates to the technical field of self-generating energy supply, and in particular to a vibration self-generating battery of a water quality low-power sensor applicable to multiple scenarios.

### BACKGROUND

In recent years, with the enhancement of environmental awareness, how to reduce the pollution of waste batteries is increasingly concerned. Meanwhile, due to the fast pace of modern life, the demands for convenient and low-cost energy supply are increasing.

In the current water quality monitoring, battery-powered sensors are often needed. However, the battery pollution to the environment is serious. In aquaculture, it is troublesome and expensive to replace the battery regularly. In addition, the expiration date of the battery is also a problem. If the battery is exhausted, the sensor cannot work normally. Therefore, it is necessary to develop a vibration self-generating battery of a water quality low-power sensor applicable to multiple scenarios.

### SUMMARY

An objective of the present disclosure is to provide a vibration self-generating battery of a water quality low-power sensor applicable to multiple scenarios, including a positive electrode magnetic cap, a battery outer cylinder, a negative electrode magnetic cap, a circuit module, a supporting component, an electromagnetic induction generating unit, and a friction nano generating unit. The positive electrode magnetic cap is arranged at an upper side of the battery outer cylinder, and the negative electrode magnetic cap is arranged at a lower side of the battery outer cylinder. The friction nano generating unit is arranged close to an inner wall of the battery outer cylinder. The electromagnetic induction generating unit is arranged at the center of the battery, and the supporting component and the circuit module are arranged below the electromagnetic induction generating unit in turn.

The battery outer cylinder is made of an insulating material, is waterproof and moisture proof.

A convex surface of the positive electrode magnetic cap is a N pole, and a concave surface of the positive electrode magnetic cap is an S pole. An inner side of the negative electrode magnetic cap is a N pole, and an outer side of the negative pole magnetic cap is an S pole.

The circuit module includes capacitors, a negative electrode copper bar, diodes, a PCB (printed circuit board), and a positive electrode magnetic bar. Two capacitors form an energy storage circuit, four diodes form a rectifying circuit, and the negative electrode copper bar and the positive electrode magnetic bar form a current steering circuit.

The supporting component includes a hollow conductive shaft, a negative electrode limiting spacer, and a positive electrode limiting spacer. The hollow conductive shaft is electrically connected to the PCB to serve as an electrode of the friction nano generating unit.

The electromagnetic induction generating unit includes a linear conductive bearing, a circular perforated magnet, and an enameled coil. The linear conductive bearing is internally provided with a ball bearing, and has conductivity with the hollow conductive shaft, and a current transmission path of the electromagnetic induction generating unit passes through the PCB, the enameled coil and the PCB in turn. The negative electrode limiting spacer and the positive electrode limiting spacer are used to limit a motion trajectory of the circular perforated magnet.

The friction nano generating unit includes a copper foil ring, a friction PTFE (Polytetrafluoroethylene) inner film, a PTFE outer diaphragm, and a friction copper foil. Another surface, with respect to the friction copper coil, of the friction PTFE inner film is wrapped with a discontinuous strip-shaped copper foil ring, and the current transmission path passes through the PCB, the copper foil ring, the friction PTFE inner film, the friction copper foil, and the PCB in turn.

As one side, close to the positive electrode magnetic cap, of the circular perforated magnet is an S pole, and one side, close to the negative electrode magnetic cap, of the circular perforated magnet is a N pole, the circular perforated magnet is kept in dynamic suspension by a repulsive force between the positive electrode magnetic cap and the negative electrode magnetic cap.

According to a power generating method of a vibration self-generating battery of a water quality low-power sensor applicable to multiple scenarios, a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film. The static electricity generates an electromotive force difference on a contact surface of the friction copper foil and the friction PTFE inner film, and a copper foil ring conducts a current generated by the electromotive force to charge a capacitor.

The circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

When a sensor is powered by a battery, a positive electrode and a negative electrode of the sensor which serves as a load are connected to a positive electrode magnetic cap and a negative electrode magnetic cap, respectively. A complete discharge current loop inside the battery passes through the negative electrode magnetic cap, a negative electrode copper bar, a PCB, a capacitor, the PCB, a positive electrode magnetic bar, a positive electrode magnetic cap, the sensor, and the negative electrode magnetic cap in turn.

The present disclosure has the beneficial effects that:

1. According to the present disclosure, magnetic line cutting generation and friction nano self-generation are achieved, the utilization rate of mechanical energy is improved, and good application prospect is achieved.

2. The power consumption of the water quality sensor can be greatly reduced, there is no need of an extra power supply and regular replacement of battery, and thus the energy source is saved, and the environment is protected.

### BRIEF DESCRIPTION OF THE DRAWINGS

FIG. 1 is a stereoscopic schematic diagram of a vibration self-generating battery of a water quality low-power sensor applicable to multiple scenarios according to the present disclosure;

FIG. 2 is a center sectional diagram of a vibration self-generating battery of a water quality low-power sensor applicable to multiple scenarios according to the present disclosure;

FIG. 3 is a schematic diagram of an electromagnetic induction generating unit according to the present disclosure;

FIG. 4 is a schematic diagram of an internal structure of an electromagnetic induction generating unit according to the present disclosure;

FIG. 5 is a schematic diagram of an unfolded structure of a friction nano generating unit according to the present disclosure;

FIG. 6 is a structural schematic diagram of a circuit module according to the present disclosure;

FIG. 7 is a structural schematic diagram of a supporting part according to the present disclosure.

### DETAILED DESCRIPTION OF THE EMBODIMENTS

The present disclosure provides a vibration self-generating battery of a water quality low-power sensor applicable to multiple scenarios, and the present disclosure is further described below with reference to the accompanying drawings and specific embodiments.

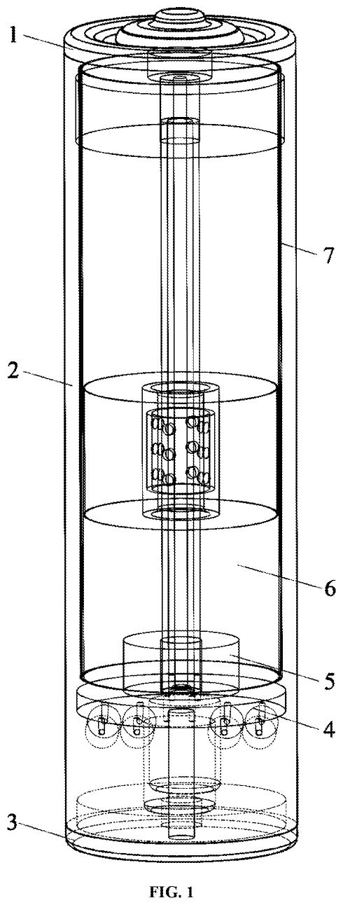

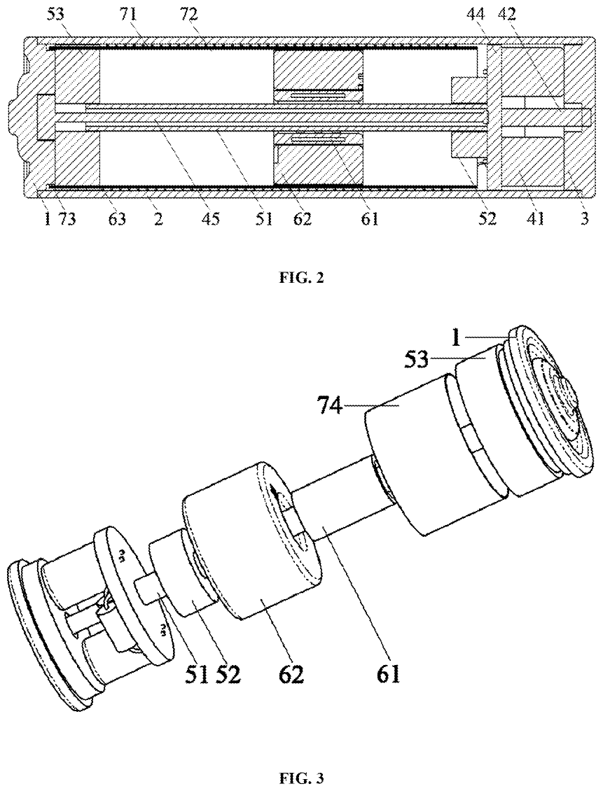

FIG. 1 is a stereoscopic schematic diagram of a vibration self-generating battery of a water quality low-power sensor applicable to multiple scenarios according to the present disclosure, including a positive electrode magnetic cap 1, a battery outer cylinder 2, a negative electrode magnetic cap 3, a circuit module 4, a supporting component 5, an electromagnetic induction generating unit 6, and a friction nano generating unit 7. FIG. 2 is a center sectional diagram of a vibration self-generating battery of a water quality low-power sensor applicable to multiple scenarios. The supporting component 5 includes a hollow conductive shaft 51, a negative electrode limiting spacer 52, and a positive electrode limiting spacer 53. The electromagnetic induction generating unit 6 includes a linear conductive bearing 61, a circular perforated magnet 62, and an enameled coil 63. The battery outer cylinder 2, as a structure support, is made of an insulating material, and is configured to connect the positive electrode magnetic cap 3 and the negative electrode magnetic cap 3, and has the characteristics of waterproof and moisture-proof A convex surface of the positive electrode magnetic cap 1 is a N pole, and a concave surface of the positive electrode magnetic cap 1 is an S pole. An inner side of the negative electrode magnetic cap 3 is a N pole, and an outer side of the negative pole magnetic cap 3 is an S pole.

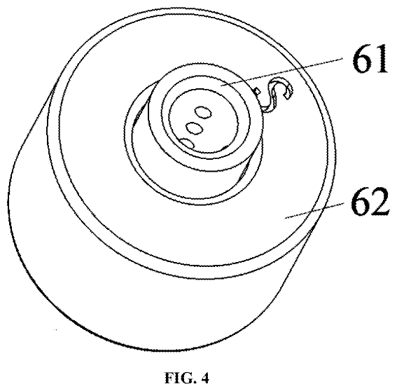

FIG. 3 is a schematic diagram of an electromagnetic induction generating unit according to the present disclosure. As one face, close to the positive electrode magnetic cap 1, of the circular perforated magnet 62 is an S pole, and one face, close to the negative electrode magnetic cap 3, of the circular perforated magnet 62 is a N pole, the circular perforated magnet is kept in dynamic suspension by a repulsive force between the positive electrode magnetic cap 1 and the negative electrode magnetic cap 3.

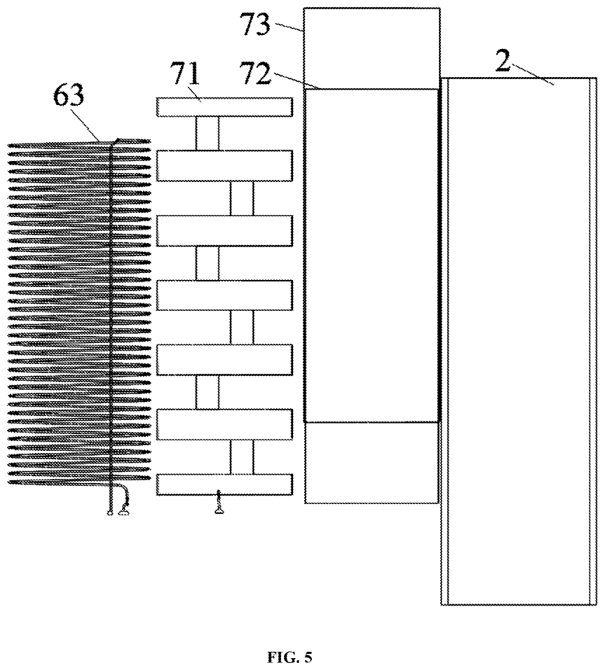

FIG. 4 is a schematic diagram of an internal structure of an electromagnetic induction generating unit according to the present disclosure. FIG. 5 is a schematic diagram of an unfolded structure of a friction nano generating unit according to the present disclosure. The electromagnetic induction generating unit 6 includes a linear conductive bearing 61, a circular perforated magnet 62, and an enameled coil 63. A current transmission path of the electromagnetic induction generating unit passes through the PCB 44, the enameled coil 63 and the PCB 44 in turn. The linear conductive bearing 61 is internally provided with a ball bearing, and has conductivity with the hollow conductive shaft 51. The hollow conductive shaft 51 is electrically connected to the PCB 44 to serve as an electrode of the friction nano generating unit 7. The friction nano generating unit 7 includes a copper foil ring 71, a friction PTFE inner film 72, a PTFE outer diaphragm 73, and a friction copper foil 74. The current transmission path of the friction nano generating unit 7 passes through the PCB 44, the copper foil ring 71, the friction PTFE inner film 72, the friction copper foil 74, and the PCB 44 in turn, respectively.

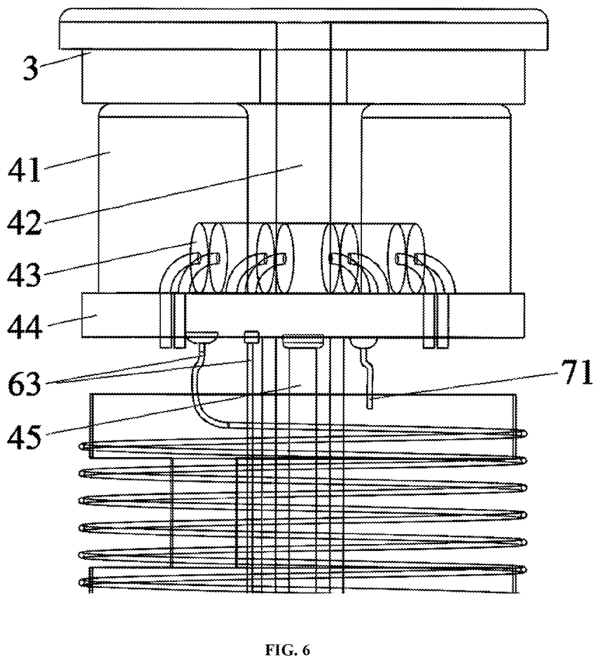

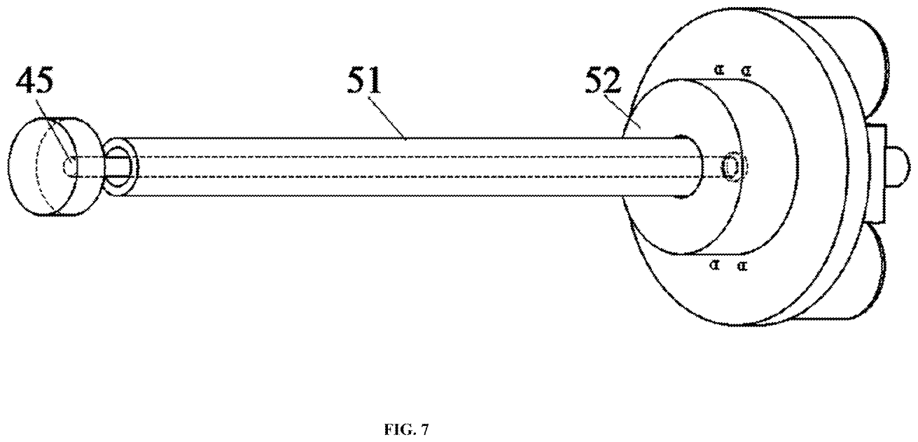

FIG. 6 is a structural schematic diagram of a circuit module according to the present disclosure. The circuit module 4 includes a rectifying circuit composed of four diodes 43, an energy storage circuit composed of two capacitors 41, and a current steering circuit composed of a negative electrode copper bar 42 and a positive electrode magnet bar 45. The rectifying circuit is used to convert an alternative current generated by friction and electromagnetic induction into a direct current, so as to charge the energy storage circuit. The current steering circuit is respectively connected to the positive and negative electrodes of the battery, so as to power an external sensor.

FIG. 7 is a structural schematic diagram of a supporting part according to the present disclosure. The positive electrode limiting spacer 53 and the negative electrode limiting spacer 52 in the supporting component 5 are used to limit a motion trajectory of the circular perforated magnet 62. Under the action of a certain vibration frequency, the circular perforated magnet 62 reciprocates up and down in the battery outer cylinder 2, and at the same time, magnetic line cutting generation and friction nano generation are achieved, the utilization rate of mechanical energy is improved, and a new solution is provided for the self-power supply of a low-power sensor in water.

The specific process of the self-generating battery is as follows:

Due to the vibration of seawater, a circular perforated magnet 62 which is originally in a balanced state is out of balance, and makes irregular reciprocating motion along a hollow conductive shaft 51, which causes a friction copper foil 74 outside the circular perforated magnet 62 to make contact with or separated from a friction PTFE inner film 72. Such a contact-separation behavior enables the friction copper coil 74 and the friction PTFE inner film 72 to generate static electricity, and then the static electricity generates an electromotive force difference on a contact surface of the friction copper foil 74 and the friction PTFE inner film 72. Another surface, with respect to the friction copper foil 74, of the friction PTFE inner film 72 is wrapped with a discontinuous strip-shaped copper foil ring 71 for conducting a current generated by the electromotive force to charge the capacitor. If the friction PTFE inner film 72 is used as a negative electrode of the friction nano generating unit 7 and the friction copper foil 74 is used as a positive electrode of the friction nano generating unit 7, a complete generating current loop is able to pass through the friction copper foil 74, the circular perforated magnet 62, the linear conductive bearing 61, the hollow conductive shaft 51, the PCB 44, the rectifying circuit, the capacitor 41, the PCB 44, the copper foil ring 71, the friction PTFE inner film 72, and back to the friction copper foil 74, respectively. Similarly, when the circular perforated magnet 62 moves towards one direction, the direction of the magnetic field changes relative to the direction of the enameled coil 63, leading to the change of a magnetic induction line in the enameled coil 63. This change may lead to the generation of a voltage across the enameled coil 63, and the direction of the voltage varies with the direction change of the circular perforated magnet 62.

When the circular perforated magnet 62 moves towards another direction, the direction of the magnetic field varies with respect to the direction of the enameled coil 63 to cause the change of the magnetic induction line in the enameled coil 63 again, thus generating a reverse voltage across the enameled coil 63. Therefore, the reciprocating motion of the circular perforated magnet 62 will generate a sine wave voltage in the enameled coil 63 to charge the capacitor 41, and a complete generating current loop is as follows: the enameled coil 63, the PCB 44, the rectifying circuit, the capacitor 41, the PCB 44, and back to the enameled coil 63.

When the sensor is powered, a positive electrode and a negative electrode of the sensor which serves as a load are connected to a positive electrode magnetic cap 1 and a negative electrode magnetic cap 3, respectively, so as to form a complete discharge loop and generate useful electric energy. The complete discharge current loop passes through the negative electrode magnetic cap 3, a negative electrode copper bar 42, the PCB 44, the capacitor 41, the PCB 44, the positive electrode magnetic bar 45, the positive electrode magnetic cap 1 and the sensor load, and returns to the negative electrode magnetic cap 3. Through this process, TENG can convert the energy generated by vibration and friction of seawater into electric energy, and then the electric energy can be used in various fields.

In the vibration self-generating battery of a water quality low-power sensor applicable to multiple scenarios, the vibration energy, after being captured, can be converted into electric energy through the principles of friction nano generation and electromagnetic induction, and used to provide electricity for the water quality sensor. Therefore, the power consumption of the water quality sensor can be greatly reduced, there is no need of an extra power supply and regular replacement of battery, a good application prospect is achieved, the energy is saved, and the environment is protected.

What is claimed is:

1. A vibration self-generating battery of a water quality low-power sensor applicable to multiple scenarios, comprising a positive electrode magnetic cap, a battery outer cylinder, a negative electrode magnetic cap, a circuit module, a supporting component, an electromagnetic induction generating unit, and a friction nano generating unit, wherein the positive electrode magnetic cap is arranged at an upper side of the battery outer cylinder, and the negative electrode magnetic cap is arranged at a lower side of the battery outer cylinder; the friction nano generating unit is arranged close to an inner wall of the battery outer cylinder; the electromagnetic induction generating unit is arranged at the center of the battery, and the supporting component and the circuit module are arranged below the electromagnetic induction generating unit in turn.

2. The vibration self-generating battery of a water quality low-power sensor applicable to multiple scenarios according to claim 1, wherein the battery outer cylinder is made of an insulating material, is waterproof and moisture proof.

3. The vibration self-generating battery of a water quality low-power sensor applicable to multiple scenarios according to claim 1, wherein a convex surface of the positive electrode magnetic cap is a N pole, and a concave surface of the positive electrode magnetic cap is an S pole; and an inner side of the negative electrode magnetic cap is a N pole, and an outer side of the negative pole magnetic cap is an S pole.

4. The vibration self-generating battery of a water quality low-power sensor applicable to multiple scenarios according to claim 1, wherein the circuit module comprises capacitors, a negative electrode copper bar, diodes, a PCB (printed circuit board), and a positive electrode magnetic bar; two capacitors form an energy storage circuit, four diodes form a rectifying circuit, and the negative electrode copper bar and the positive electrode magnetic bar form a current steering circuit.

5. The vibration self-generating battery of a water quality low-power sensor applicable to multiple scenarios according to claim 4, wherein the supporting component comprises a hollow conductive shaft, a negative electrode limiting spacer, and a positive electrode limiting spacer, wherein the hollow conductive shaft is electrically connected to the PCB to serve as an electrode of the friction nano generating unit.

6. The vibration self-generating battery of a water quality low-power sensor applicable to multiple scenarios according to claim 5, wherein the electromagnetic induction generating unit comprises a linear conductive bearing, a circular perforated magnet, and an enameled coil, wherein the linear conductive bearing is internally provided with a ball bearing, and has conductivity with the hollow conductive shaft, and a current transmission path of the electromagnetic induction generating unit passes through the PCB, the enameled coil and the PCB in turn; and the negative electrode limiting spacer and the positive electrode limiting spacer are used to limit a motion trajectory of the circular perforated magnet.

7. The vibration self-generating battery of a water quality low-power sensor applicable to multiple scenarios according to claim 4, wherein the friction nano generating unit comprises a copper foil ring, a friction PTFE (Polytetrafluoroethylene) inner film, a PTFE outer diaphragm, and a friction copper foil; another surface, with respect to the friction copper coil, of the friction PTFE inner film is wrapped with a discontinuous strip-shaped copper foil ring, and the current transmission path passes through the PCB, the copper foil ring, the friction PTFE inner film, the friction copper foil, and the PCB in turn.

8. The vibration self-generating battery of a water quality low-power sensor applicable to multiple scenarios according to claim 6, wherein as one side, close to the positive electrode magnetic cap, of the circular perforated magnet is an S pole, and one side, close to the negative electrode magnetic cap, of the circular perforated magnet is a N pole, the circular perforated magnet is kept in dynamic suspension by a repulsive force between the positive electrode magnetic cap and the negative electrode magnetic cap.

9. A power generating method of the vibration self-generating battery of a water quality low-power sensor applicable to multiple scenarios according to claim 1, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

10. A power generating method of the vibration self-generating battery of a water quality low-power sensor applicable to multiple scenarios according to claim 2, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

11. A power generating method of the vibration self-generating battery of a water quality low-power sensor applicable to multiple scenarios according to claim 3, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

12. A power generating method of the vibration self-generating battery of a water quality low-power sensor applicable to multiple scenarios according to claim 4, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

13. A power generating method of the vibration self-generating battery of a water quality low-power sensor applicable to multiple scenarios according to claim 5, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

14. A power generating method of the vibration self-generating battery of a water quality low-power sensor applicable to multiple scenarios according to claim 6, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

15. A power generating method of the vibration self-generating battery of a water quality low-power sensor applicable to multiple scenarios according to claim 7, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

16. A power generating method of the vibration self-generating battery of a water quality low-power sensor applicable to multiple scenarios according to claim 8, wherein a circular perforated magnet loses balance due to seawater vibration, and makes irregular reciprocating motion along a hollow conductive shaft, which causes a friction copper foil outside the circular perforated magnet to make contact with and separate from a friction PTFE inner film, such that static electricity is generated by the friction copper foil and the friction PTFE inner film; and the static electricity generates an electromotive force difference on a contact surface of the friction copper foil and the friction PTFE inner film, and a copper foil ring conducts a current generated by the electromotive force to charge a capacitor;
the circular perforated magnet makes reciprocating motion due to the seawater vibration, and relative motion is generated between the circular perforated magnet and the enameled coil, which makes a magnetic induction line in the enameled coil change to generate alternating electromotive force at both ends of the enameled coil, so as to charge the capacitor.

17. The power generating method of the vibration self-generating battery of a water quality low-power sensor applicable to multiple scenarios according to claim 9, wherein when a sensor is powered by a battery, a positive electrode and a negative electrode of the sensor which serves as a load are connected to a positive electrode magnetic cap and a negative electrode magnetic cap, respectively, and a complete discharge current loop inside the battery passes through the negative electrode magnetic cap, a negative electrode copper bar, a PCB, a capacitor, the PCB, a positive electrode magnetic bar, a positive electrode magnetic cap, the sensor, and the negative electrode magnetic cap in turn.

18. The power generating method of the vibration self-generating battery of a water quality low-power sensor applicable to multiple scenarios according to claim 10, wherein when a sensor is powered by a battery, a positive electrode and a negative electrode of the sensor which serves as a load are connected to a positive electrode magnetic cap and a negative electrode magnetic cap, respectively, and a complete discharge current loop inside the battery passes through the negative electrode magnetic cap, a negative electrode copper bar, a PCB, a capacitor, the PCB, a positive electrode magnetic bar, a positive electrode magnetic cap, the sensor, and the negative electrode magnetic cap in turn.

19. The power generating method of the vibration self-generating battery of a water quality low-power sensor applicable to multiple scenarios according to claim 11, wherein when a sensor is powered by a battery, a positive electrode and a negative electrode of the sensor which serves as a load are connected to a positive electrode magnetic cap and a negative electrode magnetic cap, respectively, and a complete discharge current loop inside the battery passes through the negative electrode magnetic cap, a negative electrode copper bar, a PCB, a capacitor, the PCB, a positive electrode magnetic bar, a positive electrode magnetic cap, the sensor, and the negative electrode magnetic cap in turn.

20. The power generating method of the vibration self-generating battery of a water quality low-power sensor applicable to multiple scenarios according to claim 12, wherein when a sensor is powered by a battery, a positive electrode and a negative electrode of the sensor which serves as a load are connected to a positive electrode magnetic cap and a negative electrode magnetic cap, respectively, and a complete discharge current loop inside the battery passes through the negative electrode magnetic cap, a negative electrode copper bar, a PCB, a capacitor, the PCB, a positive electrode magnetic bar, a positive electrode magnetic cap, the sensor, and the negative electrode magnetic cap in turn.
